(19) United States
(12) Patent Application Publication
(10) Pub. No.: US 2024/0296398 A1
(43) Pub. Date: Sep. 5, 2024

(54) DYNAMIC ROLES AND DYNAMIC CARE TEAM

(71) Applicant: Mobile Heartbeat, LLC, Waltham, MA (US)

(72) Inventors: Mike Kellstrand, Waltham, MA (US); Joe DiCelie, Waltham, MA (US); Sajikumar Aravind, Burlington, MA (US)

(73) Assignee: Mobile Heartbeat, LLC, Waltham, MA (US)

(21) Appl. No.: 18/593,636

(22) Filed: Mar. 1, 2024

Related U.S. Application Data
(60) Provisional application No. 63/487,719, filed on Mar. 1, 2023.

(51) Int. Cl. G06Q 10/0631

(52) U.S. Cl. CPC G06Q 10/063118

(57) ABSTRACT

In some implementations, techniques may include identifying a plurality of roster systems. Each roster system can be associated with a unit of a service provider system. In addition, the techniques may include for each roster system: accessing roster data from the roster system, each entry of the roster data having at least a time range and an identifier, the identifier having one or more of a role identifier, a location identifier, or a provider identifier; converting the roster data from a first roster configuration into a second roster configuration; and storing, by the computing device, the converted roster data in a second roster system. The implementations of these techniques including corresponding methods, devices, systems, non-transitory computer readable media, or computer program products.

### CROSS-REFERENCES TO RELATED APPLICATIONS

The present application is a non-provisional application of and claims the benefit and priority under 35 U.S.C. 119 (e) of U.S. Patent Application No. 63/487,719, filed Mar. 1, 2023, the entire contents of which are incorporated herein by reference for all purposes.

### BACKGROUND OF THE INVENTION

Recipients may receive a service over sustained time periods. These services may be administered by the staff of a service provider network called providers. Providers may change through the administration of the service, and it can be challenging to determine the current provider. Accordingly, improvements to provider identification and communication are desirable.

### BRIEF SUMMARY OF THE INVENTION

In some implementations, techniques may include identifying a plurality of roster components. Each roster component can be associated with a unit of a service provider system. In addition, the techniques may include for each roster component: accessing roster data from the roster component, each entry of the roster data having at least a time range and an identifier, the identifier having one or more of a role identifier, a location identifier, or a provider identifier; converting the roster data from a first roster configuration into a second roster configuration; and storing, by the computing device, the converted roster data in a second roster component. The implementations of these techniques including corresponding methods, devices, systems, non-transitory computer readable media, or computer program products.

### BRIEF DESCRIPTION OF THE DRAWINGS

FIG. 1 shows a block diagram of an embodiment of a provider system according to at least one example.

FIG. 2 shows a block diagram of an embodiment of a provider system according to at least one example.

FIG. 3 is a simplified sequence diagram showing a technique for consolidating roster data, according to at least one example.

FIG. 4 is a simplified sequence diagram showing a technique for transferring a critical role between user accounts, according to at least one example.

FIG. 5 is a simplified sequence diagram showing a technique for assigning a role to user accounts in an automated fashion according to at least one example.

FIG. 6 is a simplified sequence diagram showing a technique for role-based call routing according to at least one example.

FIG. 7 is a simplified sequence diagram showing a technique for associating one or more user accounts with a service recipient according to at least one example.

FIG. 8 is a simplified diagram of a process for consolidating roster data, according to at least one example.

FIG. 9 is a simplified diagram of a process for transferring a critical role between user accounts, according to at least one example.

FIG. 10 is a simplified diagram of a process for assigning a role to user accounts in an automated fashion according to at least one example.

FIG. 11 is a simplified diagram of a process for role-based call routing according to at least one example.

FIG. 12 is a simplified diagram of a process for associating one or more user accounts with a service recipient according to at least one example.

### DETAILED DESCRIPTION

The ensuing description provides preferred exemplary embodiment(s) only, and is not intended to limit the scope, applicability or configuration of the disclosure. Rather, the ensuing description of the preferred exemplary embodiment(s) will provide those skilled in the art with an enabling description for implementing a preferred exemplary embodiment. It is understood that various changes may be made in the function and arrangement of elements without departing from the spirit and scope as set forth in the appended claims.

In one example, a unit (e.g., individual practice, specialty, or facility) within a provider system (e.g., a hospital system) may manage temporal data with different roster systems (e.g., employee scheduling systems). These roster systems can be any combination of hardware and software for implementing techniques to distribute resources over a time period. Temporal data can include any data that varies over time, and temporal data can include roster data. Roster data can be a mapping of resources (e.g., service providers) to time ranges within a time period (e.g., a day, a week, a month, a year). Each roster system used on the provider system can have a unique application programming interface (API), which may lead to technical problems integrating these roster systems into a single roster that shows a complete picture of the currently allocated resources (e.g., the employees or agents of the service provider network that are currently available to provide services). To address these problems, a computing device can use an application programming interface (API) engine to pull temporal data from each roster system, and other service systems, into a single consolidated roster system. The pulled, or pushed, data is transformed from the system's native formats (e.g., a first roster configuration) into a standard format (e.g., a second roster configuration). This consolidated roster system (e.g., the second roster system) can be searched using logic-based searches, and, for example, the directory can be searched for on call physicians in a unit of a hospital (e.g., on call physicians on floor 2) or for on call physicians within a specialty (e.g., on call neurosurgeons).

Techniques described herein provide several technical advantages over provider system with multiple roster systems, and databases, for different units (e.g., practices, departments, specialties, or facilities). The single consolidated roster system described herein allows for a single consolidated roster system that can be searched more efficiently than multiple smaller roster systems. The search algorithm (e.g., search operation) used by the consolidated roster system can be optimized for larger datasets, and the half-interval search algorithm, for example, operates most efficiently on a single ordered list of data. The half-interval algorithm (e.g., half-interval operation) iteratively searches for a target, and, in each iteration, the algorithm locates a middle entry in the ordered list and compares it to the target. Based on the comparison, the half-interval operation excludes half of the ordered list that cannot contain the target. This process of excluding a half-interval of the ordered list at each round occurs until the target entry is found. In this case the dataset can be a list of service provider names in alphabetical order and the search can be for a target named “Smith, Sarah.” In the first iteration, the selected entry can be “Jimenez, Pablo” and the half-interval algorithm can determine that the target is alphabetically higher in the ordered list than the selected entry. “Smith, Sarah” is later in the alphabet than “Jimenez, Pablo,” and therefore the half-interval algorithm excludes half of the ordered list (e.g., the entries from the first entry “Anderson, Aaron” until “Jimenez, Pablo”) and selects an entry midway between “Jimenez, Pablo and the last entry in the ordered list “Watterson, David” for the next iteration. This iterative process of excluding half of the ordered list continues occurs until “Smith, Sarah” is found. An ordered list can be any dataset that is arranged in a numerical or lexicographical sequence according one or more criteria of the data. Each entry of the data can include one or more fields and the sequence of the ordered list can be based on a comparison of a particular field for each entry.

Half-interval operations are most efficient in large datasets because the data searched with each additional half-interval iteration increases logarithmically. Accordingly, a ten times increase in the search space from a consolidated database may only require a single extra iteration, while searching two databases can mean doubling the amount of iterations. Accordingly, using an application programming interface (API) engine to pull data from each roster system, and other resource systems, into a single consolidated roster system can lead to more efficient searches.

In another example, certain critical roles within a provider system, such as the cardiologist on-call, cannot be vacant because of facility policies, laws, or regulations. The problem is how to ensure that these roles do not become unintentionally vacant by the role's current occupant leaving before his/her successor is available to assume the role. These critical roles can be assigned a token and the role's occupant cannot leave the role until someone else accepts the token. The current occupant of that role can send the token to somebody else, but, if the token is rejected, the current occupant must remain in the role. Once another party accepts the token, the current occupant is removed from the role and the role passes to the token holder. Sending the token to another party can cause a pop-up on a graphical user interface (GUI) of a device controlled by the other party (e.g., user device). The pop-up can include a multiple-choice list with options such as “accept” or “reject.” If the other party does not make a selection, the pop-up can time out and default to “reject.” In some implementations, the occupant can request transfer of the role and the pop-up can be triggered by an event such as the device detecting that the other party has entered a provider facility or the pop-up can be triggered by the other party logging into the system. In some implementations, the pop up can include an option for the other party to transfer the request to someone else who is covering their shift.

Techniques described herein provide several technical advantages by reducing the risk that a critical role in the provider system goes vacant. A vacant critical role in a provider system could lead to harm to the service recipient (e.g. a patient), and tort liability, because the recipient may need care that the provider network is not equipped to provide. In addition, insurance or regulatory requirements may mandate that a provider system, such as a hospital system, maintain certain staff at all times. If these roles go vacant, the provider system may be liable for contractual penalties from the insurance provider or fines from the regulatory agency. Further, the provider system may suffer reputational damage if it is found that the system is understaffed. Accordingly, provider systems may create an auditable log of the staff in a particular role.

Resources in a provider network may be allocated to ensure that a critical role is filled. Even after the role is filled, it may be difficult to determine who currently holds the role. For example, two cardiologists may switch shifts and the switch may not be reflected in the record leading to confusion when communication with the on-call cardiologist is needed. A token-based transfer as described herein reduces the amount of effort required to track and rout calls to a critical role. In addition, the token transfers can reduce the computing resources needed to create or store an auditable log of the staff members filling a particular role, because a system implementing token-based role transfers will result in a more accurate log with a lower likelihood of gaps or false transfers.

In another example, providers may have to log into a provider roster system and assign themselves a role at the beginning of a shift and remove themselves from that role at the end of the workday. This assignment process can be error prone because it requires fallible providers to remember to assign/unassign themselves. In addition, this process is inefficient and creates extra work for providers and increases computing resources for the provider networks. Provider networks have roster systems that indicate who is assigned to a role and when. Instead of having providers assign themselves to a role, a provider system can ingest the schedule from one or more roster systems and assign/unassign roles based on which provider is scheduled to have that role at any given time.

Techniques described herein provide several technical advantages over conventional systems that require providers to assign themselves to a role. Requiring providers to log into the system and assign/unassign themselves to a role potentially exposes the system to security threats because of the large number of accounts with permission to modify the roster system. Automating scheduling can allow a provider system to reduce the number of accounts that can change the roster system because each on shift provider does not need permission to modify the roster system if assignments are automatic. Each account with permission to modify the roster system exposes the provider system to security threats, such as intentional or unintentional sabotage, because the account could be used to delete/modify an existing roster allocation.

In addition, automatic assignments can reduce the service provider network's storage and networking requirements. Without automatic assignments, staff members in the network may use a computing device to exchange messages to assign and remove themselves from a role at each shift. Each of these messages would need to be routed and processed using the provider system's computing resources. These devices would need to be authenticated before a user (e.g., a staff member) could assign or remove themselves, and authentication can involve exchanging messages and additional processing (e.g., Diffie-Helman key exchange). Accordingly, automatic assignments can reduce the provider system's computing requirements.

In another example, dynamic roles can be filled by a number of providers that vary between shifts. Determining which number to call to reach a particular dynamic role (e.g., charge nurse on floor 9) can involve searching a roster system to find which provider is currently filling that role and then locating that provider's phone number in a directory. Alternatively, some facilities may assign a particular phone to a dynamic role and the phone is passed between providers at each shift change. Using a service systems, each dynamic role can be assigned a number and a call to that number can be routed by a communication engine to a phone number for a provider currently filling that role.

Techniques described herein provide several technical advantages over current directory systems. Associating a phone number with a role, rather than a person, reduces the number of misplaced calls in work environment where errors and delays are potentially deadly. Reducing the number of misplaced calls or messages can reduce the computing resources (e.g., processing and storage resources) that are required to route communications within the provider network. In addition, provider systems can be high turnover work environments with some roles being performed by employees or agents on short term contracts (e.g., travel nurses). Such a system makes it more efficient to onboard new user devices and reduces the number of network changes caused by a provider leaving the provider system (e.g., because the phone number for a role does not change when the provider filling the role changes).

In another example, recipients are assigned providers using different roster systems that can be challenging to integrate. For example, a recipient can be assigned nursing staff using a nursing roster system and physicians can be assigned using a physician roster system. Because these roster systems are separate it can be difficult to determine the full provider team for a recipient. The provider system polls an recipient tracking system to determine which patients (e.g., recipients) are currently admitted to the provider system. Each recipient can be assigned an admission number, a medical record number, and/or a bed number. After admission, the application performance interface engine can pull the staff assigned for a recipient from each of these service systems using the admission number/medical record number/bed number and the assignments can be merged by a provider team generator into a single provider team reflecting all people assigned to a particular recipient at any given time. In addition, staff can self-assign themselves to a provider team via a user device. Once the recipient tracking systems indicates that the recipient has been discharged, the provider team can be disbanded by the provider team generator. Staff can include: nurses, physicians, physical therapists, speech therapists, occupational therapists, provider system discharge admissions administrators, child life services, social workers, etc.

Techniques described herein provide several technical advantages over current provider systems where recipients are assigned providers using different roster systems. As described above, a single large database may be more efficient to search than several distributed databases (e.g., by half-interval). In addition, integrating the recipient tracking system into the roster system can reduce the risk that the provider network has performed unnecessary operations, and stored unnecessary data, because providers are assigned to recipients who have been discharged. Similarly, the recipient tracking system can reduce the risk that there is a delay between a recipient's admission to the provider system and when the recipient is assigned a proper provider team. This delay can lead to suboptimal service for the recipient and can increase the risk of harm to the recipient and financial/reputational risk to the provider system.

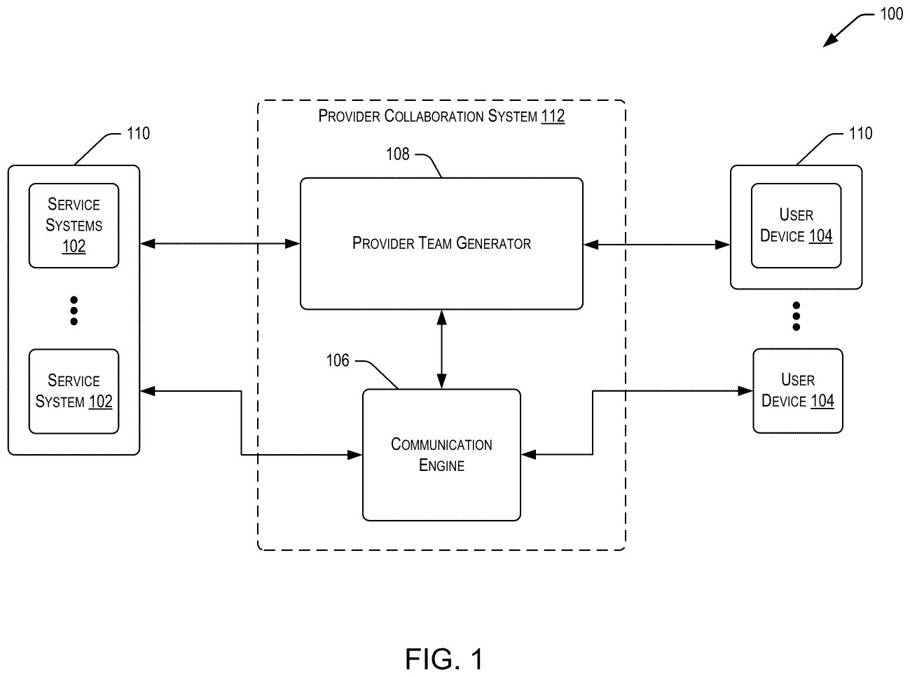

Referring first to FIG. 1, a block diagram of an embodiment of a provider system architecture 100 is illustrated, according to at least one example. The provider system architecture 100 includes a plurality of elements connected with directional arrows. The directional arrows not only indicate that the elements are connected, but also indicate the direction that data may flow with respect to the various elements. For example, data may flow between the following elements of the provider system architecture 100: a provider team generator 108 and a communication engine 106.

Generally, the provider team generator 108 is configured to collect and aggregate temporal data from systems of the provider system architecture 100 and systems outside of the provider system architecture 100. Temporal data can be a mapping between a resource of the provider system and a time range. The temporal data may identify a time range when a resource or provider is allocated to be available to provide a service. Temporal data may include a record of services that are being provided by the provider system, are going to be provided by the provider system, or that were provided by the provider system. For example, the resource can be a provider (e.g., an agent or employee of the provider system), a device, or any other entity that can be used to provide a service associated with the provider system. Once the provider team generator 108 collects and aggregates the temporal data, it may perform one or more operations with respect to the data and store it in a data store. This stored temporal data can then be accessed by components (e.g., systems) within and without the provider system architecture 100.

The temporal data is transmitted throughout the provider system architecture 100 in accordance with any suitable transmission protocol. Generally, the communication engine 106 is configured to manage the flow of such transmissions within the provider system architecture 100. Thus, the communication engine 106 receives indications of transmissions of scheduling content and tracks the origination locations of the transmissions, the destination locations of the transmissions, and any locations there between.

The provider system architecture 100 includes one or more service systems 102 and one or more user devices 104. The one or more service systems 102 are configured to share temporal data with the provider team generator 108, the communication engine 106, and each other via one or more communication networks. The one or more user devices 104 are configured to access temporal data collected by the provider team generator 108 and provide their own temporal data. Users of the one or more user devices 104 may use such temporal data to help assemble provider teams for a recipient, to accept/reject roles in a provider team, to communicate with providers, to notify providers about scheduling changes, and/or perform any other techniques relating to the management and/or administration of provider teams. The user devices 104 can be fungible user devices which are shared between providers (e.g., a mobile device that stays with the on shift intensive care unit (ICU) nurse manager). The user device 104 can be a dedicated user device that is dedicated to a particular provider (e.g., a nurse's personal phone).

While the one or more service systems 102 and the one or more user devices 104 are illustrated as communicating via the provider team generator 108 and/or the communication engine 106, this specification is not so limited. For example, each of the one or more service systems 102 may communicate with each of the one or more user devices 104 directly via other or the same communication networks. Each of the one or more service systems 102 of the provider system architecture 100 is an example of a roster system, a real time locater sensor, an recipient tracking database, an electronic health record system, or the like that can receive and/or provide temporal data as further detailed herein. Each of the one or more user devices 104 is an example of a user device that can receive and/or provide temporal data as further detailed herein. In some examples, at least some of the one or more user devices 104 may function similar to at least some of the one or more service systems 102 and vice-versa. In other words, each of the one or more user devices 104 and each of the one or more service systems 102 may both provide data and access data within the provider system architecture 100.

In some examples, the one or more service systems 102 are each associated with one or more units within the same or different provider systems. Each unit can be a subdivision of the provider system, and each unit may provide a particular service, a category of services, or one or more services in a particular geographic area or across geographic areas. In some examples, a unit can be a building of a provider system, a practice within the provider system, a floor of a provider system, a department of the provider system, etc. For example, certain ones of the one or more service systems 102 may be associated with a first unit, while other ones of the one or more service systems 102 may be associated with a second unit. Additionally, each of the one or more service systems 102 may be associated with a provider facility 110. The provider facility 110 illustrates an example of one provider facility. The provider system architecture 100, however, may include many different types of provider facilities (e.g., urgent care facilities, outpatient facilities, hospitals, clinics, and medical record service facilities) including many different types of service systems. In some examples, the one or more service systems 102 are not associated with one of the provider facilities 110, but instead are included as part of an information systems company that manages temporal data such as schedules for a patent's provider team.

The one or more service systems 102, irrespective of which unit each belongs to, may be capable of receiving, generating, processing and/or transmitting temporal data. A service system 102 can include, for example, a roster system (e.g., a system for scheduling nurses in the intensive care unit (ICU)), a sensor or detector device (e.g., a sensor for detecting whether staff (e.g., providers) are currently located at the provider facility), an electronic user device (e.g., computer, mobile device, smart phone, laptop, electronic badge, set-top box, thin client device, tablet, or pager), an electronic health record system, a server, an recipient tracking system (e.g., for tracking recipients admitted to the provider facility, an electronic tracking device (e.g., electronic tag) and/or a terminal. Examples of the one or more service systems 102 include, for example, a business and/or administrative device that can receive input from (for example) a nurse, administrator, receptionist, secretary or assistant (e.g. server, computer, mobile device, smart phone, laptop, electronic badge, set-top box, thin client device and other similar business and/or administrative devices), and other similar devices capable of generating temporal data. The one or more service systems 102 also includes entities that collect, aggregate, and store temporal data. Some of these entities may be third-parties that make medical-related data available to the provider team generator 108.

The one or more service systems 102 provide temporal data using one or more formats, some of which can be proprietary. In some examples, medical-related data from certain components is transformed, translated, or otherwise adjusted to be recognizable by the provider team generator 108.

The transmission of medical-related data from the one or more service systems 102 to the provider team generator 108 may be triggered by a variety of different events. For example, the temporal data may be transmitted periodically, upon detection of an event (e.g., upon detecting an admission of a new recipient), upon detection of an event defined by a rule (e.g., a user-defined rule), upon receiving user input triggering the transmission, or upon receiving a data request from the provider team generator 108. Each transmission can include, e.g., a single record pertaining to a single recipient or provider team; or multiple records pertaining to multiple recipients or provider teams.

In some examples, at least some of the one or more user devices 104 are associated with the provider facility 110. At least some of the one or more user devices 104 may not be associated with the provider facility 110 or any other provider facility. For example, a device can be a fungible device that is shared between authorized persons within a provider facility (e.g., a device that is shared between providers or a device that is provided to one or more recipients/beds in the provider facility), or the device can be a dedicated device that is controlled by a particular individual (e.g., a recipient or provider's personal mobile device).

Similar to the one or more service systems 102, the one or more user devices 104 may be capable of receiving, generating, processing and/or transmitting temporal data. Examples of the one or more user devices 104 include, for example, a computer, a mobile device, a smart phone, a laptop, an electronic badge, a set-top box, a thin client device, a tablet, a pager, and other similar user devices). The one or more user devices 104 may differ from the one or more service systems 102 because the one or more user devices 104 may be configured to run one or more applications developed for interacting with the temporal data collected by the provider team generator 108. For example, those user devices of the one or more user devices 104 that are not associated with the provider facility 110 (e.g., dedicated devices) may be configured to run one or more third-party applications that may rely in part on the temporal data gathered by the provider team generator 108.

Each of the one or more service systems 102 and the one or more user devices 104 may be utilized by one or more users (not shown). Each of the one or more users may be a provider that is associated with one or more units. For example, one of the one or more users can be associated with a unit as a result of being employed by the organization, physically located at a location of the organization, being an agent of the organization or receiving a service (e.g., a medical service) from the organization.

The connections between the one or more service systems 102 and the one or more user devices 104 and provider collaboration system 112, including the provider team generator 108 and the communication engine 106, are illustrated by a plurality of bi-directional arrows indicating that temporal data may flow therebetween. The temporal data flows in either direction within the provider system architecture 100 (e.g., from the provider team generator 108 and the communication engine 106 towards the one or more service systems 102 and/or the one or more user devices 104 or to the provider team generator 108 and the communication engine 106 from the one or more service systems 102 and/or the one or more user devices 104). The connections between the one or more service systems 102 and the one or more user devices 104 and the provider team generator 108 and the communication engine 106 can include any suitable network connection. A connection can be configured to support communication over a wireless medium, e.g., using Wi-Fi (IEEE 802.11 family standards), Zigbee, Bluetooth® (a family of standards promulgated by Bluetooth SIG, Inc.), Bluetooth Low Energy or other protocols for wireless data communication. In some instances, a connection can include a wired connection.

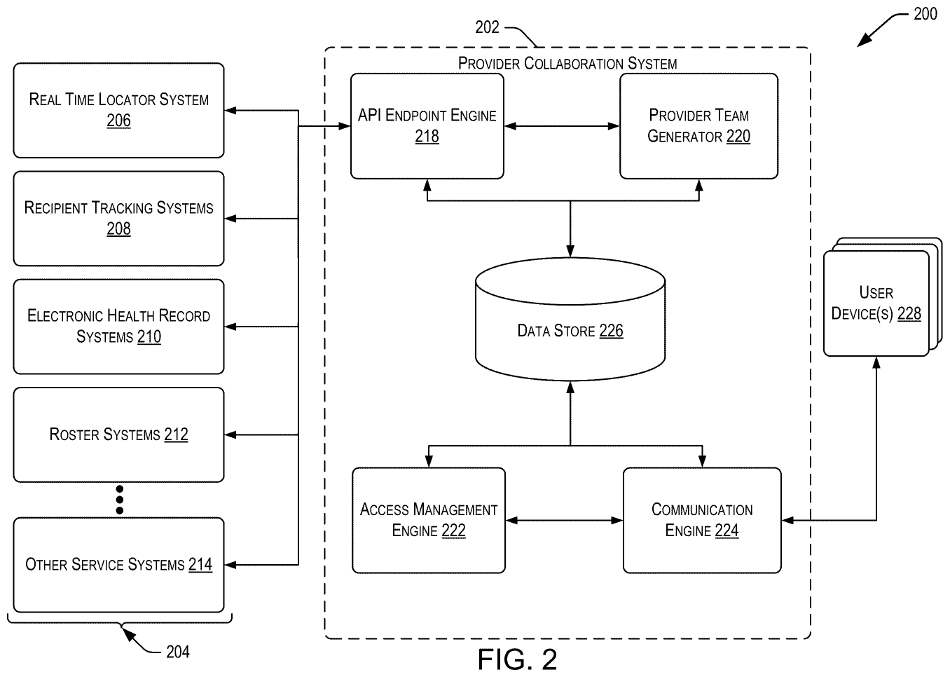

In some examples, the one or more service systems 102 and the one or more user devices 104 may communicate with the provider team generator 108 and the communication engine 106 via different information formats, different proprietary protocols, different encryption techniques, different languages, different machine languages, and the like. As will be discussed with reference to FIG. 2, the provider team generator 108 is configured to receive these many different communications from the one or more service systems 102, and in some examples from the one or more user devices 104, in their native formats and transform them into any of one or more formats. The received and/or transformed communications can be transmitted to one or more other devices (e.g., the communication engine 106, an entity device and/or a user device) and/or locally or remotely stored. In some examples, the provider team generator 108 receives medical-related data (e.g., recipient admission/discharge data) in the Health Level 7 (HL7) format or conforming to any other suitable format and/or is configured to transform received data to conform with the HL7 format.

As used herein, temporal data can include, for example, information that is created or received by a health provider, information that is created by scheduling software, and/or user-identified data. Temporal data can include information that identifies a recipient, such as personal information and/or demographic information; one or more providers associated with that recipient; and one or more calendars with assigned time periods for each associated provider. For example, the identifying information can include a recipient's name, age, sex, race, physical address, phone number, email address and/or social security number. During an assigned time period, an associated provider can provide services to the recipient (e.g., the patient) as part of a provider team.

Roster data can identify one or more providers institutions, or departments within an institution. The provider, institution, and/or department can be one associated with roles in a provider team that is providing recent or past care and/or with the patient (e.g., the recipient). For example, data can be transmitted for a recipient admitted in Hospital A and being treated by Specialist B, though the data can also identify that the recipient's primary care physician is Doctor C.

As used herein, temporal data can include protected health information, which can include individually identifiable health information that is transmitted by electronic media, maintained in electronic media, or transmitted or maintained in any other form or medium. Examples of protected health information, include, for example any information about health status, provision of health care, or payment that can be linked to a particular recipient and may include any of the following information capable of identifying the recipient: names, geographic identifiers, dates directly relating to the recipient, phone numbers, fax numbers, email addresses, social security numbers, medical record numbers, health insurance beneficiary numbers, account numbers, certificate/license numbers, vehicle identifiers and serial numbers, device identifiers and serial numbers, web Uniform Resource Locators, Internet Protocol addresses, biometric identifiers (e.g., finger, retinal, and voice prints), full face photographic images and any comparable images, and any other unique identifying number, characteristic, or code.

The one or more service systems 102 of the provider facility 110 can include and/or has access to a local or remote memory for storing generated temporal data. In some examples, the temporal data is stored by one or more servers local to the provider facility 110. Such storage may enable the provider facility 110 to retain locally temporal data pertaining to its own recipients prior to (or in conjunction with) the medical-related data being shared with the provider team generator 108 and/or the communication engine 106. In some examples, the one or more servers of the provider facility 110 share temporal data directly with a record service (not shown), and the record service makes the temporal data available to the provider team generator 108 and/or the communication engine 106. Once an electronic medical record is updated at the provider facility 110, an indication of the update may be provided to the record service. The record service may then update a corresponding record associated with the electronic medical record.

The record service can be granted access to the medical-related data generated and/or transmitted by the one or more service systems 102. In some examples, the record service includes a server, or a plurality of servers arranged in a cluster or the like. These server(s) of the record service can process and/or store temporal data generated by the one or more service systems 102. For example, one or more records can be generated for each recipient (e.g., each record corresponding to a different entity or being shared across entities). Upon receiving a communication with temporal data from a component (or provider facility), the record service can identify a corresponding record and update the record to include the temporal data (or processed version thereof). In some examples, the record service provides temporal data to the provider team generator 108.

The provider facility 110 is a facility at which care is provided to recipients. Irrespective of the type of provider facility, the provider facility 110 may provide service to recipients, update temporal data, maintain temporal data, and communicate temporal data to the provider team generator 108. At least some of the service-related data (e.g., medical-related data) may be stored local to the provider facility 110. Further, the one or more service systems 102 within the provider facility can generate temporal data including administrative information, clinical information, admission/discharge records, and financial information as part their operations within the provider facility. Examples of provider facilities include, for example, urgent care facilities, outpatient facilities, hospitals, clinics, and other suitable facilities at which care is provided to recipients.

The provider facility 110 can be an urgent care facility, an insta-care facility, an emergency room, or the like. For example, a doctor may update a particular electronic medical record of a recipient using one of the one or more service systems 102 or one of the one or more user devices 104 after receiving the recipient in the course of an emergency. In some examples, the urgent care facility may be distinct from an office of the recipient's primary provider.

The provider facility 110 can be an outpatient facility (e.g., a long-term care facility, a recovery facility, a hospice facility, a rehabilitation center, a retirement home, or the like). In some examples, the outpatient facility provide medical care (e.g., services) to patients (e.g., recipients) who are not admitted to a hospital (e.g., provider facility).

The provider facility 110 can be a hospital (e.g., a type of provider facility that provides medical, surgical, and other types of medical and nursing care). In this example, the provider facility includes one or more different wards (e.g., units) dedicated to the care and treatment of recipients with particular diseases, disorders, and the like. Within the wards, the provider facility includes a variety of different components capable of generating temporal data. The provider facility can store a portion of the generated temporal data for its own recipients locally. In some examples, users (e.g., providers, recipients, etc.) may utilize the one or more service systems 102 and/or the one or more user devices 104 to generate such temporal data.

In addition, an indication of the medical-related data can be directly or indirectly provided to the communication engine 106. Components of the provider facility can also or alternatively communicate the temporal data to the provider team generator 108 or the record service. In this manner, the provider team generator 108 has access to updated temporal data for the recipients of the service provider facility.

The provider facility 110 can be a clinic (e.g., an organization of providers that provide routine medical care). In an example, the treatment offered by the clinic is devoted primarily to outpatients. The clinic offers medical services options to populations in local communities and, in some examples, provides medical services to patients prior to the hospital providing medical services.

The provider system architecture 100 includes the one or more service systems 102 and the one or more user devices 104. One or more users (not shown) can access the service systems 102 and the user devices 104 to generate, provide, and access temporal data within the provider system architecture 100. In some examples, the temporal data may have been received by the provider team generator 108 and retained for use by others of the service systems 102 and/or the user devices 104. The one or more users can include, for example, providers, recipients, or any other suitable type of user.

In some examples, the one or more users can include providers. The providers can provide one or more medical-related services, including, for example, examination, surgery, diagnosis, consultation, counseling, scheduling of visits, handling of protected health record information, payment handling, coordination of care, management of care, and the like. In some examples, the provider is associated with the provider facility 110. In some examples, the provider is a doctor, a nurse, a surgeon, a physical therapist, a medical assistant, a facility staff person, an administrative employee, or any other person who utilizes medical-related data for treatment of recipients. In this example, the provider utilizes some of the one or more user devices 104 to send temporal data to, and/or receive from, the provider team generator 108, temporal data. In this manner, the provider can receive, or modify, scheduling assignments relating to recipients of the provider facility 110.

Referring next to FIG. 2, a block diagram of an embodiment of a provider system architecture 200 is shown, according to at least one example. The provider system architecture 200 includes a provider collaboration system 202. The provider collaboration system 202 is an example of the provider collaboration system 112 discussed with reference to FIG. 1. The provider system architecture 200 also includes one or more service systems 204. In particular, the one or more service systems 204 includes a real time locator system 206, recipient tracking systems 208, electronic health records systems 210, roster systems 212, and other service systems 214. The one or more service systems 204 are examples of the one or more service systems 102 discussed with reference to FIG. 1. Service systems can be any combination of hardware and software to perform the techniques described in relation to the service systems.

Generally, the one or more service systems 204 includes any suitable device or system capable of generating temporal data in the context of a provider system. For example, the other service system 214 may include a sensor on a door in a provider facility, and the real time locator system 206 may include a system that determines which providers are currently connected to a wireless network in a provider facility. In either case, each generation component generates some type of temporal data (e.g., data that can be used to schedule shifts for providers). For example, the temporal data provided by the sensor may be used to determine which providers have accessed a building in the provider facility and are present to assume a role in a provider team. Similarly, the temporal data provided by the real time locator system 206 may have been provided by a cardiac surgeon's dedicated user device and the temporal data can be used to determine if the provider facility is appropriately staffed (e.g., if there is a cardiac surgeon on site).

As discussed in further detail herein, temporal data generated by the one or more service systems 204 can be of a variety of formats, some of which may be proprietary. For example, a single component can generate data in multiple formats, different components can generate data in different formats, and/or different component types can result in generation of data in different formats. In some instances, formatting of a data can depend on a service having been provided, a user initiating data generation, a destination to receive the data, a location at which a service was provided, etc. Use of the provider collaboration system 202 in accordance with techniques described herein may achieve this design-making large amounts of data, in many different originating formats available to doctors, nurses, patients, administrators, and third parties, via one or more interfaces.

While the one or more service systems 204 are illustrated adjacent to each other, it is understood that each may be located within one facility or that the components may be spread out among many facilities. In addition, in some examples, the one or more service systems 204 belong to different units. One or more of the service systems may be outside of the provider system (e.g., external systems) and these external systems can provide scheduling information to the medical network provider.

Turning now to the real time locator system 206, this component includes any machine, contrivance, electronic device, sensor, or other similar related article, that is intended to aid in the determining the location of a provider. This includes, for example, electronic devices capable of implementing localization techniques such as time of flight (TOF) ranging or received signal strength indicator (RSSI) ranging techniques. These techniques can be performed by an electronic device that is capable of sending and receiving ranging messages using any suitable wireless protocol that enables data communication over a wireless medium, e.g., using near-field communication (NFC), Bluetooth Low Energy, Bluetooth® (a family of standards promulgated by Bluetooth SIG, Inc.), Zigbee, Wi-Fi (IEEE 802.11 family standards), or other protocols for wireless data communication.

The real time locator system 206 may include sensors, and, in some examples, the sensors may be configured to detect the sensors location and other details about the component or the user device. In some examples, the sensor and user device may include global positioning chips for determining a geolocation. Such geolocation information may be relevant to determining whether the sensor or user device located at a geographic location (e.g., at a particular facility). The real time locator system 206 may include one or more wireless routers (e.g., a Wi-Fi router) that provide access to a communication network. The wireless router can determine which user devices are connected to the router and this information can be used to determine whether an electronic device is present at a facility. Each of the above-listed components generates medical-related data that is provided to the provider collaboration system 202.

The recipient tracking systems 208 includes any suitable database or system that is intended to track the admission or discharge of recipients within the health care facility. The recipient tracking systems 208 can track the patent's bed/room, transfers between beds/rooms, the recipient's ward (e.g., neuro intensive care unit), transfers between wards, transfers between health care facilities in the health provider's network, or transfers from the health provider's network to an outside health provider's network. The temporal data generated by the recipient tracking systems is provided (directly or indirectly) to the provider collaboration system 202. The provided data can further include an identification of a recipient and/or other recipient-pertinent information (e.g., actual or suspected diagnosis and/or demographic information).

The electronic health record systems 210 includes any suitable computing devices used for the management and storage of electronic health records within the provider system architecture 200. The electronic health records can include notes documenting the treatment or care of recipients made by providers of the health services provider including doctors, nurses, pharmacists, occupational therapists, physical therapists, speech therapists, social workers, audiologists, and the like. The electronic health records can include consultation requests, test results, diagnoses, drug prescriptions, or any other data relevant to the treatment of a recipient within a medical facility. For example, electronic health records can include output relating to computerized physician order entry (CPOE), protected health information for recipients (i.e., a subset of medical-related data), dictations, lab results, lab requests, lab tests, orders for medical supplies, intake and checkout of recipients, medical reports, clinical tests, clinical documentation, and other similar clinical information. At least a portion of such information is provided to the provider collaboration system 202.

The roster systems 212 includes any suitable computing device, software, or database used to allocate assignments to providers. For example, the roster systems 212 is used to generate a calendar showing which providers are assigned to work during a given time period which may further include an identification of a recipient and/or other recipient-pertinent information (e.g., identification of recipients that a provider is assigned to care for). For example, the roster systems 212 is used by to assign nurses, technicians, doctors, and/or other individuals associated with a hospital, clinic, lab, or other similar entity to care for one or more recipients. At least a portion of such information is provided to the provider collaboration system 202.

Each of the one or more service systems 204 and the user device(s) 228 may include individual and/or shared storage systems, one or more processors, a user interface, a network connectivity device, and one or more ports. The storage system include memory that may be implemented, e.g., using magnetic storage media, flash memory, other semiconductor memory (e.g., DRAM, SRAM), or any other non-transitory storage medium, or a combination of media, and can include volatile and/or non-volatile media. The storage systems may also be configured to store computer-executable code or instructions for interacting with the user interface and/or for one or more applications programs, such as an application program for collecting medical-related data generated by the particular generation component.

The one or more processors may be configured to access the operating system and application programs stored within the storage systems and may also be configured to execute such program code. The one or more processors can be implemented as one or more integrated circuits, e.g., one or more single-core or multi-core microprocessors or microcontrollers, examples of which are known in the art. In operation, the one or more processors can control the operation of the particular component. The one or more processors may access and execute the program code and at any given time.

The user interface can include any combination of input and output devices. In some instances, a user can operate input devices of the user interface to invoke the functionality of the particular component or user device. For example, the user interface may enable the user to view, hear, and/or otherwise experience output from component or user device via the output devices of the user interface. Examples of output devices include a display, speakers, and the like.

The network connectivity device may enable the component or user device to communicate with the provider collaboration system 202 and other components or other user devices via one or more networks. The one or more networks may include any suitable combination of cable, cellular, radio, digital subscriber line, or any other suitable network, which may be wired and/or wireless. In some examples, the network connectivity device may enable the component or the user device to communicate wirelessly with various other components and/or the provider collaboration system 202. For example, the components may include circuitry to enable data communication over a wireless medium, e.g., using near-field communication (NFC), Bluetooth Low Energy, Bluetooth® (a family of standards promulgated by Bluetooth SIG, Inc.), Zigbee, Wi-Fi (IEEE 802.11 family standards), or other protocols for wireless data communication.

The provider collaboration system 202 includes an application programming interface (API) endpoint engine 218, a provider team generator 220, an access management engine 222, a communication engine 224, and a data store 226. Generally, the application programming interface (API) endpoint engine 218 is configured to expose endpoints that allow the collection of temporal data of different formats generated by software on the one or more service systems 204. The exposed endpoints can also be used to communicate with, or control, software on the one or more service systems 204. The application programming interface (API) endpoint engine 218 may also be configured to perform one or more operations on the collected data. For example, the application programming interface (API) endpoint engine 218 may tag data, log data, perform protocol conversion, and may support one-to-many communications. The collection may be asynchronous. In some examples, the medical-related data has been saved locally in connection with the one or more service systems 204 in many different formats having many different data structures.

From the application programming interface (API) endpoint 218, temporal data flows to the data store 226. The data store 226 (and any other data store discussed herein) may include one or more data stores, which may be distributed throughout two or more different locations (e.g., present on different devices, which can include devices of different entities and/or a cloud server). Depending on the structure of the particular data store, certain data stores may include rules for reading and writing. The data store 226 may include records, tables, arrays, and the like, which may be relational or non-relational. Depending on the data store, temporal data, records for individual recipients, results of clinical studies, business and analytics information, output data from the one or more service systems 204, and the like may be retained. The data within the data store 226 include elements or tags such that a particular data (e.g., a schedule for a single day, a provider team for a single recipient, doctor, diagnosis, type of doctor, type of treatment, recipients matching a criterion, and the like) can be retrieved.

The access management engine 222 is configured to manage access to features of the provider collaboration system 202, including access to the temporal data retained in the data store 226. For example, the access management engine 222 may verify that a user device such as user device(s) 228, which may be a dedicated user device or a fungible user device, is authorized to access the data store 226. To verify the user device(s) 228, the access management engine 222 may require that a user of the user device(s) 228 input a username and password, have a profile associated with the provider system, have paid a subscription associated with access to the data store 226, and the like. The access management engine 222 may also verify that the user device(s) 228 has an IP address or geographical location that corresponds to an authorized list, that the user device(s) 228 includes a plug-in for properly accessing the data store 226, that the user device(s) 228 is running certain applications required to access the data store 226, and the like.

The communication engine 224 is configured to retrieve the data from the data store 226 and provide one or more interfaces for interacting with elements of the provider collaboration system 202. For example, the communication engine 224 includes an interface by which an application running on user device(s) 228 can access portions of data within the data store 226 Communication engine 224 can be a version of communication engine 106 described above.

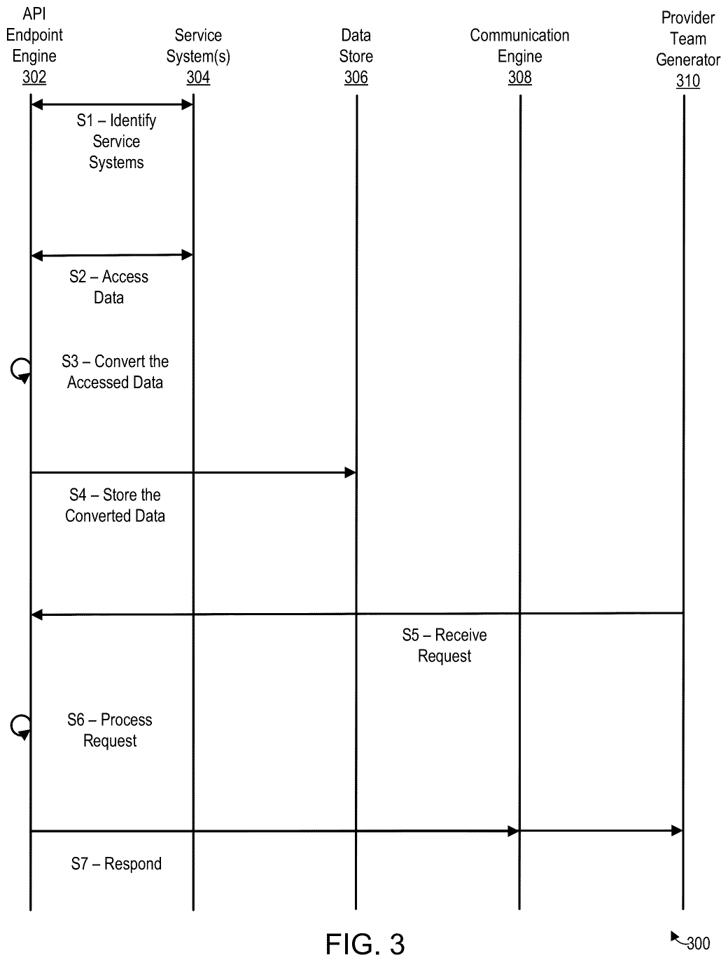

FIGS. 3, 4, 5, 6, and 7 illustrate example sequence diagrams 300, 400, 500, 600, and 700 showing respective processes as described herein. These processes depict example operations which may be performed by the various components from the system architecture 100.

FIG. 3 is a simplified sequence diagram showing a technique for consolidating temporal data, according to at least one example. Sequence diagram 300 includes API endpoint engine 302, service system(s) 304, a data store 306, a communication engine 308, and a provider team generator 310. The description of similarly named engines and modules in this disclosure can apply to the API endpoint engine 302, service system(s) 304, a data store 306, a communication engine 308, and a provider team generator 310 described with relation to sequence diagram 300.

At S1, the API endpoint engine 302 can identify one or more service system(s) 304. The service system(s) 304 can include one or more of a real time locator system, a recipient tracking system, an electronic health record system, a roster system, or any other service system. The service system(s) 304 can be associated with one or more units, and the API endpoint engine 302 can identify the service system(s) 304 by querying each service system for a list of units associated with that service system. In some embodiments, the API endpoint engine 302 can identify one or more units (e.g., by a unique identifier) and each service system 304 can confirm whether any of the units are in a list of associated units for the system. The API endpoint engine 302 can use the list of units associated with each service system 304 to identify the service systems. Some or all of the service system(s) 304 can push a list of associated units to the API endpoint engine 302 at regular intervals (e.g., daily) or in response to an event (e.g., the addition of a new service system). In some embodiments, the service system(s) 304 can be identified regardless of any associated units.

At S2, the API endpoint engine 302 can access data from the service system(s) 304 identified at S1. In some embodiments, accessing the data can mean that the service system(s) 304 send data to the API endpoint engine 302 in response to a query at S1 or S2. For example, the query can request confirmation that a particular service system is associated with one or more units. If the service system is associated with any of the units, the service system can return data corresponding to the associated units in the response. The service system(s) 304 can provide data for some or all of the systems at S2. In some embodiments, the service system(s) 304 can push data to the API endpoint engine 302 at regular intervals (e.g., weekly) or in response to an event (e.g., a change in the system's data). In some embodiments, the API endpoint engine 302 can register to receive any updates to the data of a particular service system.

At S3, the API endpoint engine 302 can convert the data accessed at S2. The data can be received at the API endpoint engine 302 in the format that the data was stored in a service system (e.g., a native format). Each entry in the data can include a time range and an identifier. The time range can be one or more point in time, one or more intervals of time, or a combination of both. The data accessed at S2 can be temporal data such as roster data. The roster data can be a schedule of assignments identifying when providers will be available to provide services, where those services will be provided, and the recipients that are to receive the service. The roster data can include information about the providers that are currently providing services or are currently available to provide services.

Converting the data accessed at S2 can mean changing the data from a first format to a second format. For example, dates in the first format can be stored in a mm/dd/yy configuration and converting the data can mean changing the dates to a yyyy/mm/dd configuration. Converting the data can mean combining data from two different service system(s) 304. For example, the data from a first service system can have two fields per entry and the data from a second service system can have three fields per entry. The two service systems can have one common field that can be used to determine a correspondence between entries in both sets of data, and, for example, the common field could be a recipient identifier. The combined data can include the common field and one or more fields from each of the combined datasets. For example, the combined entries for the data from the first and second service system described above can include four fields (e.g., the common field, a field from the first service system and two fields from the second service system). In some embodiments, the data from multiple service systems can be combined.

At S4, the API endpoint engine 302 can store (e.g., record) the converted data from S3. The data can be stored in a data store 306 (e.g., a second roster system). In some embodiments, the data can be stored in one or more of the service system(s) 304, and the data may or may not be stored in the data store 306. Storing the data can mean sorting the entries in the data into at least one ordered list according to at least one field of the data. The order in the ordered list can be a numerical order or a lexicographical order.

At S5, a request can be received at the API endpoint engine 302. A request originating from a user device can be received at the API endpoint engine 302 via a communication engine 308. The request from the user device can be a request to route a message to a target device. The request can be received from a provider team generator 310 in some embodiments. The request can include a target time and a target identifier. The target time can be one or more points in time, one or more time ranges, or a combination of both. The target identifier can be information that identifies a recipient, a provider, a unit, a role, a provider team, etc. The request can be a request to route one or more messages to a target user device (e.g., a target device), and the request can be a query to identify the target user device.

At S6, the request received at S5 can be processed by the API endpoint engine 302. Processing the request can include performing a search of the data store 306. The search can be a half-interval operation as described herein. The search can be performed by the data store 306 without input from the API endpoint engine 302 in some embodiments.

At S7, the API engine 302 can provide a response to the request from S5. The response can include returning the requested information to the source of the request. For example, a request from a user device, that was received via communication engine 308, can be returned to the user device. In some embodiments, the request from the user device can be a request to route a message to a target device. In such embodiments, the response can be used by the communication engine 308 to route the message and the response may not be returned to the user device that generated the request. In some embodiments, the request can be returned to the provider team generator 310.

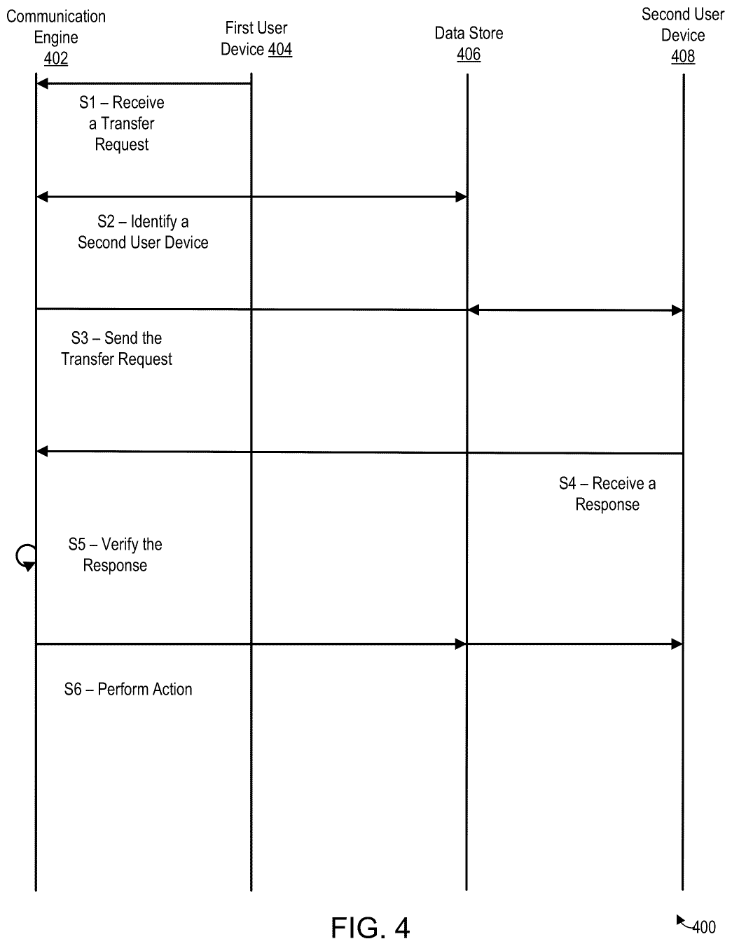

FIG. 4 is a simplified sequence diagram showing a technique for transferring a critical role between user accounts, according to at least one example. Sequence diagram 400 includes a communication engine 402, a first user device 404, a data store 406 and a second user device 408. The description of similarly named engines and modules in this disclosure can apply to the communication engine 402, the first user device 404, the data store 406 and the second user device 408 described with relation to sequence diagram 400.

At S1, a transfer request from a first user device can be received at the communication engine 402. The transfer request can be received from a first user device 404, and the transfer request may be received via a network connection. The first user device 404 can be associated with a first user account (e.g., a provider identifier) and the second user device 408 can be associated with a second user account. The request can include a request to transfer an active token associated with a role that is occupied by the first user account. The request can identify a role and a second user account, and the second user account can be the intended successor for the role occupied by the first account.

Each user account can have a private key that is used to create the transfer request. The transfer request can include the active token, and, to make the request, the first user device 404 can append information about the proposed transaction to the token and sign the appended token with the private key for the first user account. The appended information can include information identifying the role, information identifying the second user account, and a timestamp. In this way, the token can be a verifiable record of transactions involving the token. The information identifying the second user account can be a public key associated with the second user account. The active token

At S2, the communication engine 402 can provide the request to the data store 406 for information to identify a second user device associated with the second user account. The user devices may be fungible user devices and the second account identified in the request may be associated with different user devices at different times. The communication engine 402, or the data store 406, can use the information in the request from S1 to identify the second user device 408 using information from data store 406. The second user device 408 can be a user device on which the second user account is currently authenticated (e.g., the second user account is signed into the second user device). The data store 406 may include public keys associated with each user account, and these public keys can be used to identify each user account. For example, the data store 406 can include a mapping between public keys, user accounts, and device identifiers. The data store 406 can provide information including this mapping to the communication engine 402. In some embodiments, this mapping can be stored on the communication engine 402. The communication engine 402 may use the public key corresponding to the first user account to verify the first user's signature of the request (e.g., by calculating the secret generated by the signature). The information identifying the second user device 408 can be a phone number, a device identifier, a network address, etc.

At S3, the communication engine 402 can send the transfer request to the second user device identified at S2. The request can be a copy of the request sent to the data store 406. In some embodiments, the data store 406 can verify the request and return the verified request at S2. In such embodiments, the verified request can be forwarded from the first user device 404 to the second user device 408. The second user device 408 can verify the request using a public key corresponding to the first user account, and the second user device 408 may retrieve the public key from data store 406.

At S4, a response from the second user device 408 can be received at the communication engine. The response can include information identifying acceptance of the transfer request, rejection of the request, or a request to delay the transfer (e.g., wait 30 min). in some embodiments, the response can be information indicating that the second user device 408 was unable to verify the request using the public key. The second user device 408 may accept the transfer request by signing the transfer request (e.g., the token) with a private key corresponding to the second user account. The accepted transfer request may also include a signed timestamp corresponding to when the second user device signed the request.

At S5, the communication engine 402 can verify the response from the second user device 408 that was received at S4. If the response is unsigned, then the response is not verified. If the response was signed, the communication engine 402 can use the public key for the second user device to verify the signature.

At S6, the communication engine 402 can perform an action in response to the result of the verification at S5. If the response was verified at S5, the communication engine 402 can instruct the data store 406, or provider team engine, to transfer the role from the first user account to the second user account. The instruction can include the token that was signed by both user accounts, and data store 406 may use the public keys for the first user account and second user account to verify the signatures before transferring the token. If the response at S4 included a request to delay the transfer, the communication engine 402 may resend the request to the second user device 408 after a delay.

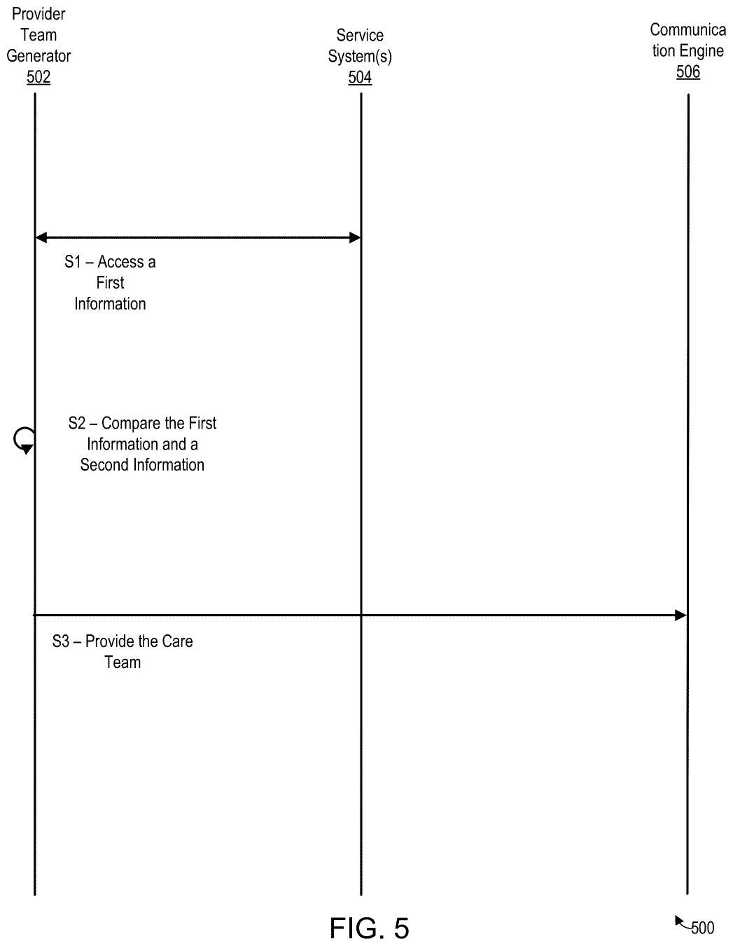

FIG. 5 is a simplified sequence diagram showing a technique for assigning a role to user accounts in an automated fashion according to at least one example. Sequence diagram 500 includes a provider team generator 502, service system(s) 504, and communication engine 506. The description of similarly named engines and modules in this disclosure can apply to the provider team generator 502, service system(s) 504, and communication engine 506 described with relation to sequence diagram 500.

At S1, a first information can be accessed by the provider team generator 502. Accessing the information can include sending a request from the provider team generator 502 to one or more service system(s) 504. The request can identify a recipient (e.g., by a recipient identifier), and the one or more service system(s) 504 can locate and return the first information in response to the request. The first information can be requested by the provider team generator 502 at regular intervals, in response to an event. In some embodiments, the first information can be pushed to the provider team generator by the service system(s) 504 at regular intervals or in response to an event (e.g., a change to data at a service system). For example, the first information can be pushed from a service system (e.g. a real time locator system) if a roster system determines that a provider has ended their shift.

The first information can include different information from different service systems. For example, the first information can include a recipient location that is received from a recipient tracking system. The recipient location can identify a unit, building, room, bed, or coordinates where a patient is located. The first information can include a current roster that includes information identifying one or more providers that are scheduled to provide services as to the identified recipient. The first information may also include information identifying the locations of one or more user devices associated with the one or more identified providers. This information can be received from a real time locator system. The provider team generator can filter this first information so that the first information includes providers that are currently scheduled to provide a service associated with the recipient and are located within a threshold distance of the recipient location.

At S2, the provider team generator 502 can compare the first information and a second information. The second information can be the providers that are currently in the provider team for the recipient identified in the request at S1. The provider team can include one or more roles and a provider can be assigned to each role. For example, a provider team may include the following roles: attending physician, nutritionist, and orderly. The provider team generator 502 may compare the first information and the second information to assign a provider to each role. In some embodiments, the first information and the second information can identify services that are currently being provided to the recipient or services that are scheduled to be provided to the recipient within a threshold time period. For example, an electronic health record system can provide the services, and the care team generator can identify one or more roles for each service. Accordingly, the first information and the second information can each contain one or more roles for the recipient and comparing the first information and the second information can include determining roles that are needed to provide current or scheduled services to the recipient. The care team generator 502 can update the provider team to include the determined roles.

If a provider is identified in the first information but not the second information, the provider team generator 502 can add the provider to the recipient's provider team. If a provider is identified in the second information but not the first information, the provider can be removed from the provider team. In some embodiments, the providers identified in the first information can be designated as the provider team. In some embodiments, location information for the providers identified in the second information can be retrieved from a real time location service. In such embodiments, each role in the provider team can be assigned to a provider that eligible to fill the role and who is closest to the recipient location. The role for each participant can be included in the roster information.

At S3, the care team generator 502 can provide the care team from S2 to a communication engine 506. The communication engine 506 can use the provider team to route messages between members of the provider team. The messages can include text messages, video messages, audio messages, audio calls, video calls, etc. The communication engine 506 can provide notifications to the provider team in response to changes to one or more of the service systems 504. For example, the provider team may be notified if a recipient tracking system indicates that the recipient has been moved within the service system.

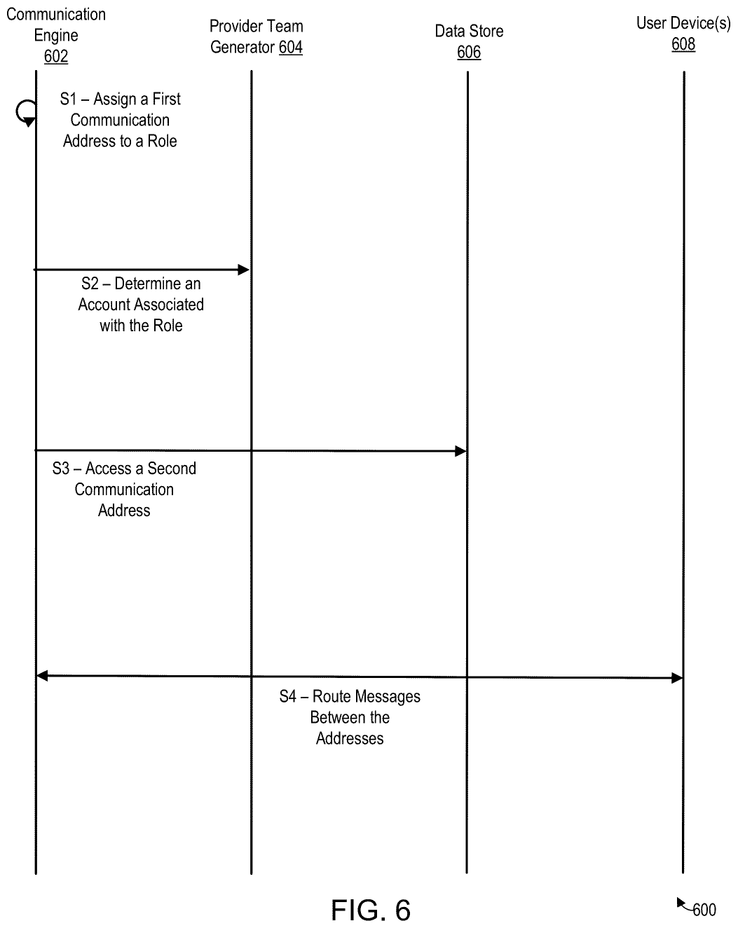

FIG. 6 is a simplified sequence diagram showing a technique for role-based call routing according to at least one example. Sequence diagram 600 includes a communication engine 602, a provider team generator 604, a data store 606, and user device(s) 608. The description of similarly named engines and modules in this disclosure can apply to the communication engine 602, the provider team generator 604, the data store 606, and the user device(s) 608 described with relation to sequence diagram 600.

At S1, a first communication address can be assigned to a role by a communication engine 602. The first communication address and the second communication address can be a phone number, a uniform resource locator, an internet protocol address, an email address, and the like. The first communication address can be assigned to a role in a provider team database in data store 226 or the role can be assigned within the communication engine 602. The role can be associated with information identifying a recipient or information identifying a provider team. The communication engine 602 can receive calls/messages at the first communication address and forward those messages to one or more additional communication addresses or user devices.

At S2, an account associated with the role can be determined by the communication engine 602. The account can be a provider identifier or any other information that can be used to identify a provider. The communication engine 602 can request the account from the provider team generator 604, or the provider team generator 604 can provide the account to the communication engine. A request to the care team generator 604 can include information identifying the role, recipient, or provider team from S1. Determining the account can mean retrieving a unique identifier associated with the account from the provider team database. The unique identifier associated with the account can be retrieved using the information identifying the role, recipient, or provider team.

At S3, the communication engine 602 can access a second communication address from a data store 606. In some embodiments, the communication engine 602 can access the second communication address within the communication engine. The second communication address can be an address for a user device 608 that is associated with the account at S2. The user device can be a personal device of the provider associated with the account at S2. In some embodiments, the user device 608 can be a fungible user device that is shared between accounts. A fungible user device can be associated with the account if the account is currently logged into the device (e.g., authenticated on the device).

At S4, the communication engine 602 can route messages between the first address and the second address. The messages can be routed between the addresses by the communication engine 602. A message from a first user device can be received at the communication engine. This message can be intended for a role and the message can be addressed to the first address. The communication engine 602, in response to receiving the message, can forward the message to the second address. In some embodiments, the account filling the role can change and a third address can be determined using the same techniques that were used to determine the second address. In some embodiments, techniques to determine the third address can be performed in response to a determination that sending the message to the second address has failed (e.g., the account is no longer logged into the user device associated with the second address),

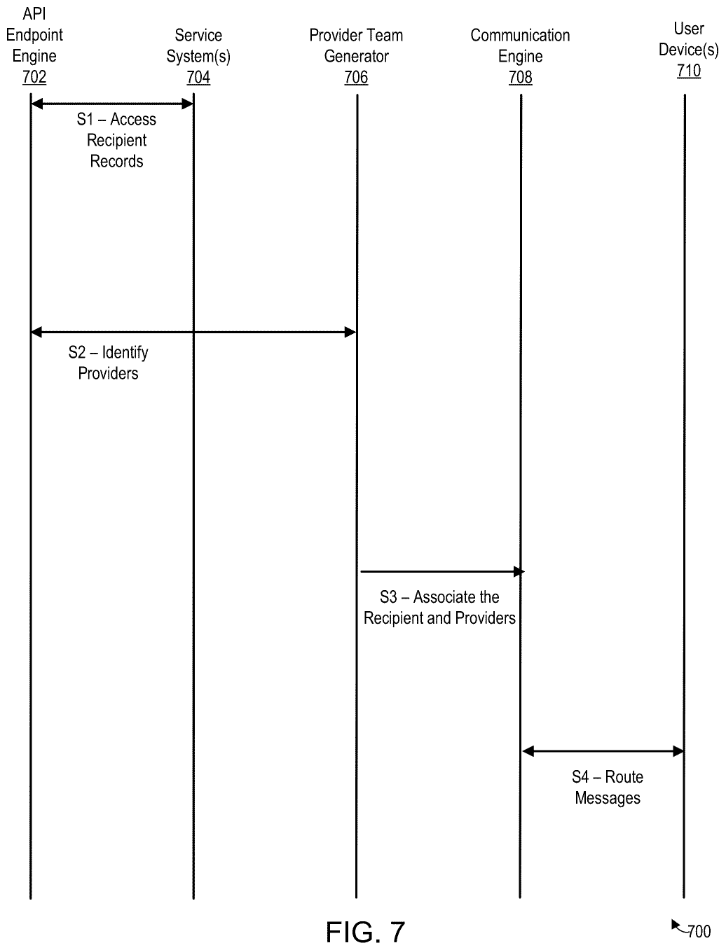

FIG. 7 is a simplified sequence diagram showing a technique for associating one or more user accounts with a service recipient according to at least one example. Sequence diagram 700 includes API endpoint engine 702, service system(s) 704, a provider team generator 706, a communication engine 708, and user device(s) 710. The description of similarly named engines and modules in this disclosure can apply to the API endpoint engine 702, the service system(s) 704, the provider team generator 706, the communication engine 708, and the user device(s) 710. described with relation to sequence diagram 700.

At S1, the API endpoint engine 702 can access one or more recipient records from one or more service system(s) 704. For example, the API endpoint engine 702 can request records (e.g., recipient records) from a recipient tracking system. These records can be sent to the API endpoint engine 702 in response to a request, at regular intervals, or in response to an event (e.g., a new recipient being admitted). The recipient records can be the records of one or more of the recipients who are currently admitted to a service facility.

At S2, the provider team generator 706 can use the recipient records from S1 to identify providers. The identified providers can be some or all of a provider team, and identifying the providers may include generating a provider team. In some embodiments, identifying the providers can include verifying that the provider team is up to date, and identifying the provider team may include adding or removing a provider form the provider team.

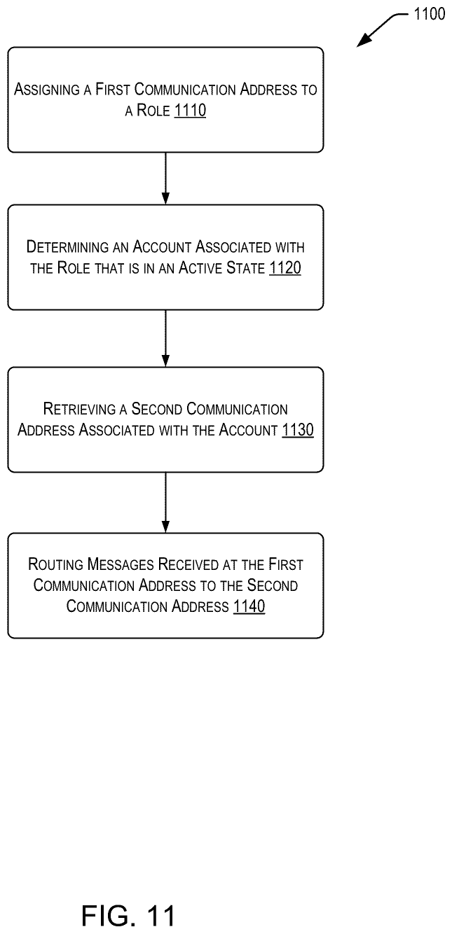

At S3, the communication engine 708 can associate the recipient from S1 with the providers identified at S2. The communication engine may associate the recipient and providers in response to a request from the provider team generator 706, or in response to an attempt by any device to send a message to a member of the recipient's care team. Associating the recipient and providers can include associating an account corresponding to the recipient with an account corresponding to the providers. Associating the accounts may include using any of the techniques of this disclosure for routing messages to roles (e.g., the techniques described with regard to FIGS. 6 and 11). For example, associating the accounts may include assigning an address for a role in the recipient's provider team to the participant.

At S4, the communication engine 708 can route messages to one or more user devices 710. The user devices can be fungible user devices that are associated with the providers identified at S2. The messages can be text messages, video messages, audio messages, image files, video files, text files, video over internet protocol (VOIP) calls, cellular network calls, or any other communication message. Routing the messages may include using any of the techniques of this disclosure for routing messages to roles (e.g., the techniques described with regard to FIGS. 6 and 11).

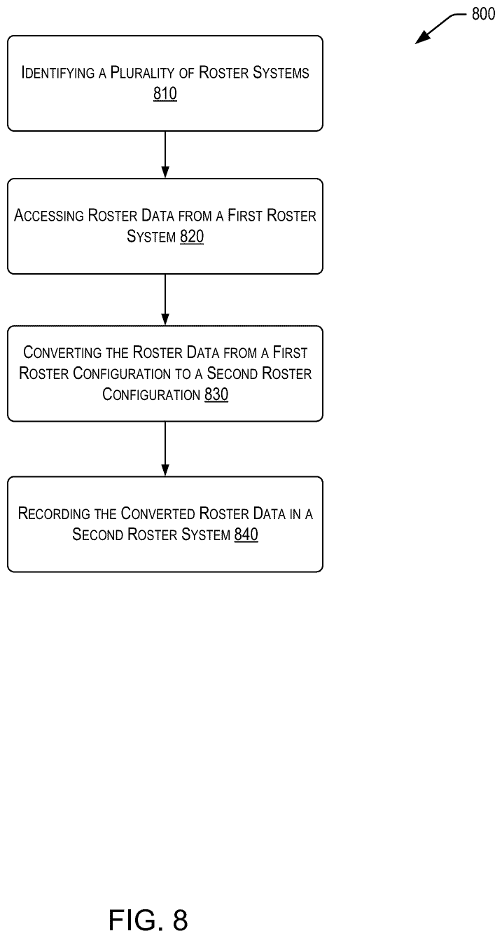

FIGS. 8, 9, 10, 11, and 12 illustrate example flow diagrams showing respective processes 800, 900, 1000, 1100, and 1200 as described herein. These processes are illustrated as logical flow diagrams, each operation of which represents a sequence of operations that can be implemented in hardware, computer instructions, or a combination thereof. In the context of computer instructions, the operations represent computer-executable instructions stored on one or more non-transitory computer-readable storage media that, when executed by one or more processors, perform the recited operations. Generally, computer-executable instructions include routines, programs, objects, components, data structures, and the like that perform particular functions or implement particular data types. The order in which the operations are described is not intended to be construed as a limitation, and any number of the described operations can be omitted or combined in any order and/or in parallel to implement the processes.

Additionally, some, any, or all of the processes may be performed under the control of one or more computer systems configured with executable instructions and may be implemented as code (e.g., executable instructions, one or more computer programs, or one or more applications) executing collectively on one or more processors, by hardware, or combinations thereof. As noted above, the code may be stored on a computer-readable storage medium, for example, in the form of a computer program comprising a plurality of instructions executable by one or more processors. The computer-readable storage medium is non-transitory.

FIG. 8 is a simplified diagram of a process 800 for consolidating roster data (e.g., temporal data), according to at least one example. This process can involve an application programming interface (API) that can access data from different roster systems (e.g., a plurality of first roster systems), transform the data into a standardized format, and store the data to a consolidated temporal database (e.g., a second roster system, a provider team directory, etc). The consolidated database can be used to assemble provider teams for a recipient without having to consult multiple databases to locate providers. The process 800 is illustrated as a logical flow diagram, each operation of which can be implemented in hardware, computer instructions, or a combination thereof. In the context of computer instructions, the operations may represent computer-executable instructions stored on one or more computer-readable storage media that, when executed by one or more processors, perform the recited operations. Generally, computer-executable instructions include routines, programs, objects, components, data structures and the like that perform particular functions or implement particular data types. The order in which the operations are described is not intended to be construed as a limitation, and any number of the described operations can be combined in any order and/or in parallel to implement the process.

Turning to process 800 in greater detail, at block 810, the process 800 includes the provider collaboration system 202 identifying a plurality of first roster systems. The first roster systems can be service systems, such as service systems 102, or service systems 204, and the roster systems can be identified by a provider collaboration system (e.g., provider collaboration system 112, provider collaboration system 202). Each service system can be configured to manage temporal data (e.g., roster data) for a unit of a provider system (e.g., provider facility 110, medical provider organization, and the like). Roster data can include scheduling data for providers or any other temporal data. This scheduling data can indicate time ranges when a provider is available to provide a service.

At block 820, the process 800 includes accessing roster data from a first roster system. The roster system can be a service system, and the service systems can include real time locator system 206, recipient tracking systems 208, electronic health record systems 210, roster systems 212, or other service systems 214. The roster system can be accessed via a network connection. The roster data can be requested by application programming interface (API) engine 218. Accessing the roster data can include receiving the roster data in response to a request. In some embodiments, accessing the roster data can include receiving the temporal data at a scheduled time or in response to the occurrence of an event. Blocks 820-840 can be performed for each roster system identified at block 810.

At block 830, the roster data can be converted from a first roster configuration to a second roster configuration. The first roster configuration can be the format of the data at the service system and the second roster configuration can be a second format for the second roster system. In some embodiments, the first roster configuration can be the second roster configuration for at least one of the service systems. The temporal data can be translated from the first format to the second format by the application programming interface (API) engine 218. The temporal data can be accessed at 820 in the native formats for each service system (e.g., accessed via a network connection). The API engine 218 can map the data from each native format to the standard format. This mapping can include translating the information and structure of the stored data. For example, one native format may have a name field that is “Last Name, First Name” a second format may be “last name, first name, gender” and a third format may be “FIRST NAME LAST NAME.” The information in the name field for each of these formats is not mutually intelligible, and, in the case of the second format, the structure of the information in the format is different because the name field also includes gender. Translating the data could mean that the API engine 218 turns all of these formats into “LAST NAME, FIRST NAME” and separates the gender information from the second format into a separate “Gender” category.

At block 840, the converted roster data can be recorded in a second roster system. The second roster system can be a searchable database (e.g., data store 226). The searchable database can be a database such as the data store 226. The converted roster data can be indexed by a unique identifier for a recipient of the provider system. Recording the temporal data can include storing the data in a computer readable format in a non-transitory computer readable medium.

In some embodiments, a user device(s) 228 can access the second roster data in the second roster system (e.g., data store 226) via communication engine 224. The second roster data can be accessed by a user device, such as user device 104 or user device(s) 228. The second roster data can be accessed from a data store 226 and provided to the user device by a communication engine (e.g., communication engine 106 or communication engine 224).

Permission to access the second roster data can be determined by an access management engine 222. The accessed temporal data can be used by the communication engine 224 to generate a graphical display or a graphical user interface that is shown on the user device(s) 228. The graphical display, or user interface, can be used to present the roster data (e.g., temporal data, scheduling data, etc.) to one or more providers, to allow the providers to alter the roster data, or to receive requests for particular scheduling assignments/requests for time off from the provider. The provider team generator 220 can use the temporal data to generate a roster (e.g., a schedule) for a period of time (e.g., a daily schedule, a weekly schedule, a monthly schedule, a yearly schedule). The roster data in data store 226 can be used by access management engine 222 to control access to the provider collaboration system 202 based on whether the profile attempting to access the data is scheduled to work (e.g., provide services) during the attempted access. In addition, assignments in the roster system can be used by the access management engine 222 to control access to recipient data based on the assignments (e.g., a provider may only be able to access electronic health records for recipients under the provider's care).

The roster data can be used to route a message from a user device to a target device. For example, the user device can be a first member of a provider team and the target device can be a second member of the provider team. The converted roster can be accessed so that messages are routed to current provider team members. Accessing the converted roster data can include receiving a target time and a target identifier from a user device (e.g., user device(s) 228). The target time can be a point in time, a range of times, the current time, etc. The target identifier can be an identifier associated with a recipient (e.g., a provider team identifier, a bed number, a recipient identifier, an electronic heath record number, recipient demographic information, etc.) One or more of the target time and the target identifier can be used to identify one or more target entries (e.g., roster data entries). The messages can be video messages, audio messages, audiovisual messages, text-based messages, image messages, audio calls, audiovisual calls, etc.

A half-interval operation can be performed to locate a target entry in the second roster system. The converted roster data in the second roster system can be sorted into an ordered list. The order can be lexicographical order (e.g., alphabetical order by name) or a numeric order (e.g., an ascending order based on an eight-digit recipient identifier). Each entry in the converted roster data can include one or more fields (e.g., one or more fields of data) and the converted roster data can be sorted into an ordered list based on any one of the fields.

The half-interval operation can be performed iteratively, and, at each iteration, an operation entry can be identified in an operation set. At the first iteration, the operation set can be the entire ordered list. The operation entry can be a randomly selected from the ordered list or the operation entry can be selected based on the position of an operation entry in the ordered list (e.g., a midpoint of the ordered list). The operation entry's position can be determined with the field that was used to sort the entries into the ordered list (e.g., the sorting field). Once selected, the operation entry can be compared to the target entry, and the half-interval operation is concluded if the target entry and the operation entry match.

If the entries do not match, the ordered list is divided, using the operation entry, into a first set of entries and a second set of entries (e.g., the operation entry is contiguous with both sets). The sorting field for the target entry and the operation entry can be compared to determine if the target entry is in the first set of entries or the second set of entries. The operation entry is contiguous with both sets of entries, and one set of entries will be higher in the order than the operation entry and the other set of entries will be lower in the order. The comparison of the target entry and operation entry can be used to determine the relative positions of the two entries (e.g., the target entry is higher in the order than the operation entry), and this relative position can be used to determine which set of entries contains the target entry (e.g., the first set of entries is higher in the order than the operation entry and the first set of entries must contain the target entry). The set of entries that is determined to contain the target entry can be designated as the operation set for the next iteration. This iterative process continues until the target entry is located.

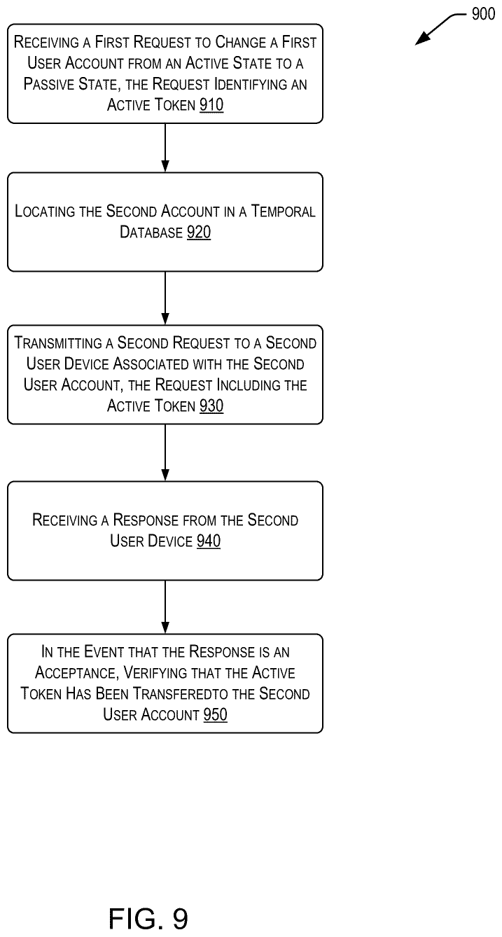

FIG. 9 is a simplified diagram of a process 900 for transferring a critical role between user accounts, according to at least one example. The process 900 can be used to reduce the possibility that a critical role, such as the on-call neurosurgeon, is left unintentionally vacant. Ownership of a non-fungible token is used to indicate which account is currently in the critical role. The active account, with the token, cannot become passive until the token is transferred to another account that assumes the active role. The process 900 is illustrated as a logical flow diagram, each operation of which can be implemented in hardware, computer instructions, or a combination thereof. In the context of computer instructions, the operations may represent computer-executable instructions stored on one or more computer-readable storage media that, when executed by one or more processors, perform the recited operations. Generally, computer-executable instructions include routines, programs, objects, components, data structures and the like that perform particular functions or implement particular data types. The order in which the operations are described is not intended to be construed as a limitation, and any number of the described operations can be combined in any order and/or in parallel to implement the process.

Turning to process 900 in greater detail, at block 910, a first communication engine 224 of a provider collaboration system 202 can receive a first request. The first request being a request to change the first user account from an active state to a passive state and the first request identifying a second user account as a successor. The first account can be an account that is currently occupying a role in a provider team and the first request can identify an active token associated with the role. Identifying the active token can mean that a copy of the active token is included in the request. Ownership of the active token can be determined using a temporal database (e.g., data store 226). The token can be a unique identifier that is signed with a private key for the current account that is actively occupying the role associated with the active token. The temporal database can contain a list of active tokens that are linked to roles in a provider system and determining the ownership of the account can mean determining which account is associated with the private key that has been used to sign the token most recently. The request can be generated by the first account, the second account, a service system, an external system, etc.

At block 920, the communication engine 224 can locate a second user device, associated with the second account, in the temporal database. The second user device can be located by locating the second user account using a unique identifier for the first account or the second account. The temporal database can be one or more databases in a data store such as data store 226. Locating the second user device can include locating an address associated with the second account or the second user device (e.g., an internet protocol address, a uniform resource locator, a phone number, an email address). In some embodiments, the communication engine 224 can locate a public key associated with the second account in addition to locating an address associated with the second account.

At block 930, the communication engine 224 can transmit a second request to a second user device that is associated with the second user account. The second request can be a request to change the second user account from the passive state to the active state. The second user device can be a computer device such as user device 104 or one of the user device(s) 228. In some examples, the request may include a copy of the active token from 910.

At block 940, the communication engine 224 can receive a response from the second user device associated with the second user account. The response can be a response to the request from 930 and the response can indicate an acceptance, a rejection, or a delay (e.g., a response indicating that the second user device would like to delay the decision for 15 minutes). In some examples, an acceptance can be a response that includes a copy of the token that is signed with the private key for the second user device.

At block 950, the communication engine 224 can verify that the active token has been transferred the second account in the event that the response is an acceptance. Transferring ownership of the token can indicate that the second account has assumed the role and the first account can be changed to a passive state. The token can include a unique identifier for the role that is signed with a private key associated with the account that currently in an active state for the role. Transferring the active token can mean verifying that the token received at 940 was signed with the private key for the successor account (e.g., the private key for the second account). Upon receiving a response at 940, the communication engine 224 can check that the response includes a signed active token and the communication engine can verify ownership of the token using one or more public keys that are associated with one or more accounts in the temporal database. The active token is owned by the account associated with the public key that most recently signed the active token. Only one account can own the active token at any one time because the token can only be signed with one public key. A signature may include a timestamp that is signed with the public key and the signed timestamp can be used to determine which account most recently signed the active token. Transferring the active token changes the first account from an active state to a passive state and changes the second account from a passive state to an active state.

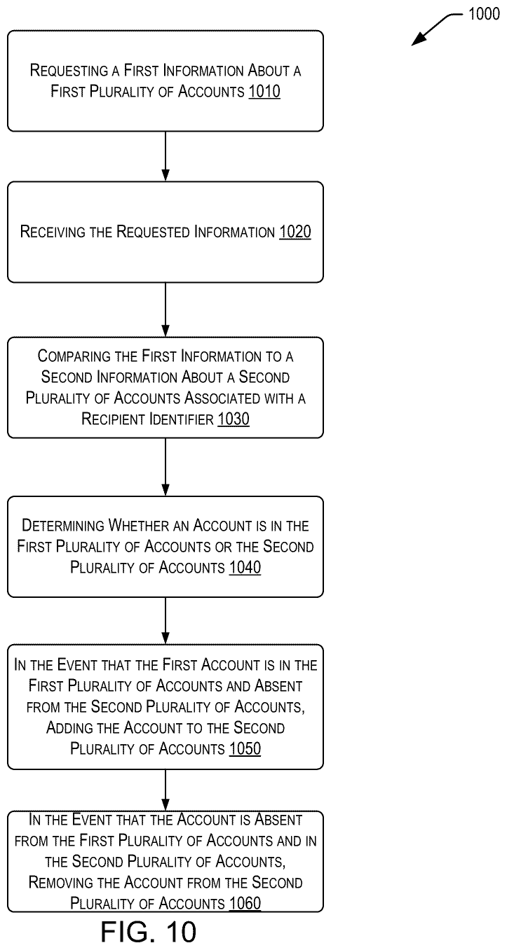

FIG. 10 is a simplified diagram of a process 1000 for assigning a role to providers in an automated fashion according to at least one example. A service system's providers are currently required to log into a roster system and assign themselves to a role in the provider team. This assignment process can be automated to improve the security of the service system's roster system by limiting access to the roster system. The process 1000 is illustrated as a logical flow diagram, each operation of which can be implemented in hardware, computer instructions, or a combination thereof. In the context of computer instructions, the operations may represent computer-executable instructions stored on one or more computer-readable storage media that, when executed by one or more processors, perform the recited operations. Generally, computer-executable instructions include routines, programs, objects, components, data structures and the like that perform particular functions or implement particular data types. The order in which the operations are described is not intended to be construed as a limitation, and any number of the described operations can be combined in any order and/or in parallel to implement the process.

Turning to process 1000 in greater detail, at block 1010, a provider team generator 108 can request a first information about a first plurality of accounts from a service system 102. The first information can be a list of unique identifiers for one or more accounts that are associated with a recipient identifier and currently scheduled to be active (e.g., the first plurality of accounts are scheduled to work during the current time period). The service system can be one or more of service system 102 or service system 204. In some implementations, the service system can be multiple service systems. The request can be generated by a computer device (e.g., computing device) such as user device 104, user device(s) 228, provider collaboration system 112, or provider collaboration system 202. The request can be made to multiple service systems in some embodiments. The service system can be a roster system such as roster system 212.

At block 1020, the provider team generator 108 can receive the information requested at 1010 from the service system 102. The information can be received at a computer device such as user device 104, user device(s) 228, provider collaboration system 112, or provider collaboration system 202. The information can be received from the one or more service systems via a network connection.

At block 1030, the provider team generator 108 can compare the first information to a second information about a second plurality of accounts that are associated with the recipient identifier in a provider team database (e.g., data store 226, provider team generator 220, etc.). Comparing the accounts can mean that a unique identifier for each account is compared to determine if there is a difference between the accounts in the first plurality of accounts and the second plurality of accounts. The second plurality of accounts can be accounts that are currently assigned to a provider team for a recipient associated with the recipient identifier. The provider team database can be a database such as data store 226.

At block 1040, the provider team generator 108 can determine whether an account is in the first plurality of accounts or the second plurality of accounts. An account that is in the first plurality of accounts is an account that is associated with the recipient identifier and scheduled to work during the current shift. An account that is present in the second plurality of accounts is an account that is currently a member of a provider team for the recipient associated with the recipient identifier.

At block 1050, the provider team generator 108 can add the account from 1030 to the second plurality of accounts if the provider team generator 108 determines that the account is present in the first plurality of accounts and absent from the second plurality of accounts. An account that is present in the first plurality of accounts but not the second plurality of accounts is an account that is scheduled to work with a recipient but that has not been added to the provider team (e.g., the second plurality of accounts).

At block 1060, the provider team generator 108 can remove the account from 1030 from the second plurality of accounts if the account is absent from the first plurality of accounts and present in the second plurality of accounts. An account that is present in the second plurality of accounts but not the first plurality of accounts is an account that is not currently scheduled to work with a recipient but is still added to the provider team (e.g., the second plurality of accounts).

FIG. 11 is a simplified diagram of a process 1100 for role-based call routing according to at least one example. Communication within a provider system or provider facility can be simplified by a system that routes calls based on who is currently occupying a role. Instead of having to find the phone number for a particular provider that is currently occupying a role, the system can route the call to whichever provider currently holds that role. The process 1100 is illustrated as a logical flow diagram, each operation of which can be implemented in hardware, computer instructions, or a combination thereof. In the context of computer instructions, the operations may represent computer-executable instructions stored on one or more computer-readable storage media that, when executed by one or more processors, perform the recited operations. Generally, computer-executable instructions include routines, programs, objects, components, data structures and the like that perform particular functions or implement particular data types. The order in which the operations are described is not intended to be construed as a limitation, and any number of the described operations can be combined in any order and/or in parallel to implement the process.

Turning to process 1100 in greater detail, at block 1110, a communication engine 224 can assign a first communication address to a role. The address can be assigned by a computer device such as user device 104, user device(s) 228, provider collaboration system 112, or provider collaboration system 202. The communication address can be a phone number, a uniform resource locator, an internet protocol address, an email address, and the like. The address can be assigned to a role in a provider team database in data store 226. The communication engine 224 can receive calls/messages at the first communication address and forward those messages to one or more additional communication addresses or user devices.

At block 1120, the communication engine 224 can check the provider team generator 220 to determine an account associated with the role from 1110. The account can be an account that is in an active state (e.g., currently assigned to provide a service) and associated with the role. Determining the account can mean retrieving a unique identifier associated with the account from the provider team database.

At block 1130, the communication engine 224 can retrieve a second communication address from a directory database in data store 226. The account can be retrieved using a unique identifier for the account retrieved from the provider team database at 1120. The directory database can be one or more databases in data store 226. The second communication address can be a communication address for a personal mobile device associated with the account from 1120.

At block 1140, messages received at the first communication address can be routed to the second communication address. The messages can be routed by communication engine 106 or communication engine 224. The sender of the messages addresses the messages to the first communication address, for the role, and the communication engine 224 can route the messages to the second communication address (e.g., the address of the mobile device of the provider currently occupying the role).

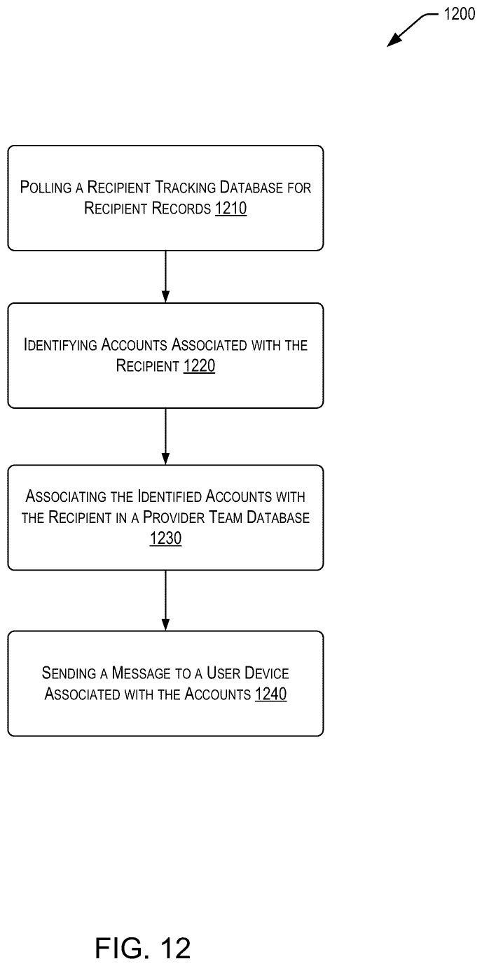

FIG. 12 is a simplified diagram of a process 1200 for assigning a provider team to an admitted recipient according to at least one example. Instead of assigning recipients to a provider team using different service systems, a centralized system can use recipient tracking records to automatically assign staff to a recipient's provider team. The process 1200 is illustrated as a logical flow diagram, each operation of which can be implemented in hardware, computer instructions, or a combination thereof. In the context of computer instructions, the operations may represent computer-executable instructions stored on one or more computer-readable storage media that, when executed by one or more processors, perform the recited operations. Generally, computer-executable instructions include routines, programs, objects, components, data structures and the like that perform particular functions or implement particular data types. The order in which the operations are described is not intended to be construed as a limitation, and any number of the described operations can be combined in any order and/or in parallel to implement the process.

Turning to process 1200 in greater detail, at block 1210, an API endpoint engine can poll recipient tracking systems 208 for a plurality of admitted recipient records. Each admitted recipient record can be associated with a recipient, and the admitted recipient record can comprise at last one of a bed identifier, a medical record identifier, or an admission identifier. The recipient tracking system 208 can be polled by a computer device such as user device 104, user device(s) 228, provider collaboration system 112, or provider collaboration system 202. The recipient tracking system 208 can be one or more databases such as data store 226 or the admissions data. The admitted recipient records can be forwarded from the API endpoint engine 218 to the provider team generator 220.

At block 1220, the provider team generator 220 can identify one or more accounts (e.g., information identifying a provider) in a provider team generator 220 or the data store 226 using the recipient records from 1210. The one or more accounts can be associated with providers that the provider team generator 220 has assigned as members of a provider team corresponding to the recipient from 1210. Identifying the one or more accounts can mean generating a care team. For example, a care team generator can be identified using any of the techniques for generating a care team disclosed herein (e.g., techniques described with regard to FIGS. 5 and 10). The one or more accounts can be identified by the provider team generator 220 comparing at least one of the bed identifier, the medical record identifier, or the admission identifier to entries in the provider team database (e.g., the provider team generator 220).

At block 1230, the provider team generator 220 can associate the one or more accounts identified in 1220 with the recipient in a provider team database in data store 226. The provider team database can be one or more databases such as data store 226. Associating the one or more accounts can mean adding a provider team identifier to a provider team field for each accounts entry in the provider team database.

At block 1240, the communication engine 224 can send a message to a user device 228. The user device can be a user device associated with the identified one or more accounts from 1220. The message can be sent by a communication engine such as communication engine 106 or communication engine 224. The accounts can be accounts that are part of a provider team and the message can identify the recipients that the provider team is treating. The recipients can be identified by name, bed identifier, medical record identifier, or admissions identifier.

Specific details are given in the above description to provide a thorough understanding of the embodiments. However, it is understood that the embodiments may be practiced without these specific details. For example, circuits may be shown in block diagrams in order not to obscure the embodiments in unnecessary detail. In other instances, well-known circuits, processes, algorithms, structures, and techniques may be shown without unnecessary detail in order to avoid obscuring the embodiments.

Implementation of the techniques, blocks, steps and means described above may be done in various ways. For example, these techniques, blocks, steps and means may be implemented in hardware, software, or a combination thereof. For a hardware implementation, the processing units may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, other electronic units designed to perform the functions described above, and/or a combination thereof.

Also, it is noted that the embodiments may be described as a process which is depicted as a flowchart, a flow diagram, a swim diagram, a data flow diagram, a structure diagram, or a block diagram. Although a depiction may describe the operations as a sequential process, many of the operations can be performed in parallel or concurrently. In addition, the order of the operations may be re-arranged. A process is terminated when its operations are completed, but could have additional steps not included in the figure. A process may correspond to a method, a function, a procedure, a subroutine, a subprogram, etc. When a process corresponds to a function, its termination corresponds to a return of the function to the calling function or the main function.

Furthermore, embodiments may be implemented by hardware, software, scripting languages, firmware, middleware, microcode, hardware description languages, and/or any combination thereof. When implemented in software, firmware, middleware, scripting language, and/or microcode, the program code or code segments to perform the necessary tasks may be stored in a machine readable medium such as a storage medium. A code segment or machine-executable instruction may represent a procedure, a function, a subprogram, a program, a routine, a subroutine, a module, a software package, a script, a class, or any combination of instructions, data structures, and/or program statements. A code segment may be coupled to another code segment or a hardware circuit by passing and/or receiving information, data, arguments, parameters, and/or memory contents. Information, arguments, parameters, data, etc. may be passed, forwarded, or transmitted via any suitable means including memory sharing, message passing, token passing, network transmission, etc.

For a firmware and/or software implementation, the methodologies may be implemented with modules (e.g., procedures, functions, and so on) that perform the functions described herein. Any machine-readable medium tangibly embodying instructions may be used in implementing the methodologies described herein. For example, software codes may be stored in a memory. Memory may be implemented within the processor or external to the processor. As used herein the term “memory” refers to any type of long term, short term, volatile, nonvolatile, or other storage medium and is not to be limited to any particular type of memory or number of memories, or type of media upon which memory is stored.

Moreover, as disclosed herein, the term “storage medium” may represent one or more memories for storing data, including read only memory (ROM), random access memory (RAM), magnetic RAM, core memory, magnetic disk storage mediums, optical storage mediums, flash memory devices and/or other machine readable mediums for storing information. The term “machine-readable medium” includes, but is not limited to portable or fixed storage devices, optical storage devices, and/or various other storage mediums capable of storing that contain or carry instruction(s) and/or data.

While the principles of the disclosure have been described above in connection with specific apparatuses and methods, it is to be clearly understood that this description is made only by way of example and not as limitation on the scope of the disclosure.

What is claimed is:

1. A computer-implemented method, comprising:
identifying, by a computing device, a plurality of first roster systems, each first roster system associated with a unit of a provider system; and
for each first roster system:
accessing by the computing device, a roster data of the first roster system, each entry of the roster data comprising at least a time range and an identifier, the identifier comprising one or more of a role identifier, a location identifier, or a provider identifier;
converting, by the computing device, the roster data from a first roster configuration into a second roster configuration; and
recording, by the computing device, the converted roster data in a second roster system.

2. The method of claim 1, further comprising:
accessing, by the computing device, the converted roster data.

3. The method of claim 2, wherein accessing the converted roster data comprises:
receiving, by the computing device, a target time and a target identifier from a user device;
identifying, by the computing device, a target entry using the target time and the target identifier, wherein the target entry is associated with a target device; and
routing, by the computing device, a message from the user device to the target device.

4. The method of claim 1, wherein the roster data of the first roster system is accessed via a network connection.

5. The method of claim 1, further comprising:
sorting, by the computing device, the converted roster data into an ordered list, wherein a field of each entry of the converted roster data is sorted into a numerical order or a lexicographical order, the each entry of the converted roster data comprising one or more fields of data.

6. The method of claim 5, further comprising:
performing, by the computing device, a half-interval operation to locate a target entry in the ordered list.

7. The method of claim 6, wherein performing the half-interval operation comprises, iteratively performing until the target entry is located in the ordered list:
identifying, by the computing device, an operation entry in an operation set comprising one or more entries of the ordered list;
dividing, by the computing device, the operation set into a first set and a second set, wherein the operation entry separates the first set and the second set, wherein the first set and the second set are each a plurality of contiguous entries in the ordered list and the operation entry is contiguous with the first set and the second set;
determining, by the computing device, that the target entry is in the first set; and
designating, by the computing device, the first set as the operation set.

8. A computing device, comprising:
one or more memories; and
one or more processors in communication with the one or more memories and configured to perform operations to:
identify a plurality of first roster systems, each first roster system associated with a unit of a provider system; and
for each first roster system:
access a roster data of the first roster system, each entry of the roster data comprising at least a time range and an identifier, the identifier comprising one or more of a role identifier, a location identifier, or a provider identifier;
convert the roster data from a first roster configuration to a second roster configuration; and
record the converted roster data in a second roster system.

9. The device of claim 8, wherein the one or more processors are further configured to perform operations to:
access the converted roster data.

10. The device of claim 9, wherein accessing the converted roster data comprises operations to:
receive a target time and a target identifier from a user device;
identify a target entry using the target time and the target identifier, wherein the target entry is associated with a target device; and
route a message from the user device to the target device.

11. The device of claim 8, wherein the roster data of the first roster system is accessed via a network connection.

12. The device of claim 8, wherein the one or more processors are further configured to perform operations to:
sort the converted roster data into an ordered list, wherein a field of each entry of the converted roster data is sorted into a numerical order or a lexicographical order, the each entry of the converted roster data comprising one or more fields of data.

13. The device of claim 12, wherein the one or more processors are further configured to perform operations to:
perform a half-interval operation to locate a target entry in the ordered list.

14. The device of claim 13, wherein the operations to perform the half-interval operation comprise performing, iteratively until the target entry is located in the ordered list, operations to:
identify an operation entry in an operation set comprising one or more entries of the ordered list;
divide the operation set into a first set and a second set, wherein the operation entry separates the first set and the second set, wherein the first set and the second set are each a plurality of contiguous entries in the ordered list and the operation entry is contiguous with the first set and the second set;
determine that the target entry is in the first set; and
designate the first set as the operation set.

15. A non-transitory computer-readable medium storing a plurality of instructions that, when executed by one or more processors of a computing device, cause the one or more processors to perform operations to:
identify a plurality of first roster systems, each first roster system associated with a unit of a provider system; and
for each first roster system:
access a roster data of the first roster system, each entry of the roster data comprising at least a time range and an identifier, the identifier comprising one or more of a role identifier, a location identifier, or a provider identifier;
convert the roster data from a first roster configuration into a second roster configuration; and
record the converted roster data in a second roster system.

16. The non-transitory computer-readable medium of claim 15, wherein the instructions cause the one or more processors to perform further operations to:
access the converted roster data.

17. The non-transitory computer-readable medium of claim 16, wherein accessing the converted roster data comprises operations to:
receive a target time and a target identifier from a user device;
identify a target entry using the target time and the target identifier, wherein the target entry is associated with a target device; and
route a message from the user device to the target device.

18. The non-transitory computer-readable medium of claim 15, wherein the instructions cause the one or more processors to perform further operations to:
sort the converted roster data into an ordered list, wherein a field of each entry of the converted roster data is sorted into a numerical order or a lexicographical order, the each entry of the converted roster data comprising one or more fields of data.

19. The non-transitory computer-readable medium of claim 18, wherein the instructions cause the one or more processors to perform further operations to:
perform a half-interval operation to locate a target entry in the ordered list.

20. The non-transitory computer-readable medium of claim 19, wherein the operations to perform the half-interval operation cause the one or more processors to perform, iteratively until the target entry is located in the ordered list, operations to:
identify an operation entry in an operation set comprising one or more entries of the ordered list;
divide the operation set into a first set and a second set, wherein the operation entry separates the first set and the second set, wherein the first set and the second set are each a plurality of contiguous entries in the ordered list and the operation entry is contiguous with the first set and the second set;
determine that the target entry is in the first set; and
designate the first set as the operation set.
